(19) United States
(12) Patent Application Publication
(10) Pub. No.: US 2008/0152157 A1
(43) Pub. Date: Jun. 26, 2008

(54) Method and system for eliminating noises in voice signals

(75) Inventors: Zhongsong Lin, Beijing (CN); XiaoCheng Wang, Beijing (CN); YuHong Feng, Beijing (CN); Hao Deng, Beijing (CN)

Correspondence Address: SILICON VALLEY PATENT AGENCY, 7394 WILDFLOWER WAY, CUPERTINO, CA, 95014, US

(73) Assignee: Vimicro Corporation

(21) Appl. No.: 11/614,088

(22) Filed: Dec. 21, 2006

(51) Int. Cl. A61F 11/06

(52) U.S. Cl. 381/71.1; 381/94.1

(57) ABSTRACT

Techniques for an adaptive filter method are disclosed. According to one aspect of the techniques. According to one aspect of the present invention, an adaptive filter technique is disclosed. The operation of an adaptive filter in one embodiment involves the following operations: estimating a noise value according to coefficients w(i) of the adaptive filter and a current frame of a reference noise signal u(n); subtracting estimated noise from the current frame of a voice signal s(n) mixed with noise to get one frame of pure voice signal e(n); providing the current frame of pure voice signal e(n) to the adaptive filter; calculating an adaptive step size and estimating a voice probability contained in the current frame of reference noise; adjusting the adaptive step size according to the voice probability contained in the current frame of reference noise; refreshing the adaptive filter coefficient according to the current frame of the pure voice signal, the current frame of reference noise signal, the adjusted adaptive step size, and then returning to the operation of filtering a next frame of voice mixed with noise according to the refreshed adaptive filter coefficient until the voice signal mixed with noise is completely processed.

### BACKGROUND OF THE INVENTION

1. Field of the Invention

The present invention relates to voice signal processing, and more particularly to method and system for eliminating noises mixed in voice signals.

2. Description of Related Art

Generally, NLMS (Normalized Least Mean Squares) algorithm is a familiar adaptive filter algorithm for eliminating noises mixed in voice signal. FIG. 1 is a block diagram showing schematically how to eliminate noises by an NLMS adaptive filter. In this process, two microphones are needed, it is assume that the voice mixed with the noise is recorded by one microphone being away from a speaker and near the noise source, and a reference noise is recorded by the other microphone being away from the noise source and near to the speaker. An adaptive filter estimates the noise mixed in the voice according to the reference noise. By a subtracter, the noise estimated by the adaptive filter is subtracted from the voice with mixed noise to obtain a pure voice. Finally, the pure voice is provided to the adaptive filter as a feedback signal.

There are a number of ways to implement an NLMS algorithm. However, some involve extensive computations while others would reduce the magnitude of the filtered voice or introduce echo.

Thus, there is a need for efficient techniques to enhance the voice quality in a device that is implemented with an NLMS adaptive filter.

### SUMMARY OF THE INVENTION

This section is for the purpose of summarizing some aspects of the present invention and to briefly introduce some preferred embodiments. Simplifications or omissions in this section as well as in the abstract or the title of this description may be made to avoid obscuring the purpose of this section, the abstract and the title. Such simplifications or omissions are not intended to limit the scope of the present invention.

In general, the present invention pertains to improved method of implementing for adaptive filter. According to one aspect of the present invention, an adaptive filter technique is disclosed. The operation of an adaptive filter in accordance with the present invention involves the following operations:

gathering frames of the voice signal s(n) mixed with noise and frames of the reference noise u(n);

inputting a current frame of the reference noise signal u(n) to the adaptive filter;

estimating a noise value according to coefficients w(i) of the adaptive filter and the current frame of the reference noise signal u(n);

subtracting the estimated noise from the current frame of the voice signal s(n) mixed with noise to get one frame of pure voice signal e(n) by a subtracter;

providing the current frame of pure voice signal e(n) to the adaptive filter;

calculating an adaptive step size μ and estimating a voice probability contained in the current frame of reference noise, wherein μ is a constant, or μ=c/Eₙ, c is constant, Eₙ is an estimated energy of the current frame of the reference noise signal;

adjusting the adaptive step size μ according to the voice probability contained in the current frame of reference noise. Specifically, the higher the probability is, the smaller the adaptive step size μ becomes;

refreshing the adaptive filter coefficient according to the current frame of the pure voice signal, the current frame of reference noise signal, the adjusted adaptive step size μ̅; and

finally, returning to the operation of filtering a next frame of voice mixed with noise according to the refreshed adaptive filter coefficient until the voice signal mixed with noise is completely processed.

One of the objects, features, and advantages of the present invention is to provide an adaptive filter that may be used to minimize noise in audio/voice signals.

Other objects, features, and advantages of the present invention will become apparent upon examining the following detailed description of an embodiment thereof, taken in conjunction with the attached drawings.

### BRIEF DESCRIPTION OF THE DRAWINGS

These and other features, aspects, and advantages of the present invention will become better understood with regard to the following description, appended claims, and accompanying drawings where:

FIG. 1 is a block diagram schematically showing how to eliminate noises by adaptive filter;

FIG. 2 is a flowchart specifically showing how to eliminate noise via an NLMS adaptive filter;

FIG. 3 is a block diagram showing an adaptive filter system according to one embodiment of the present invention;

FIG. 4 is a flowchart or process of adaptively removing noise from a voice signal according to one embodiment of the present invention;

FIG. 5 shows one example of a step size controller used in FIG. 3; and

FIG. 6 shows another example of a step size controller used in FIG. 3.

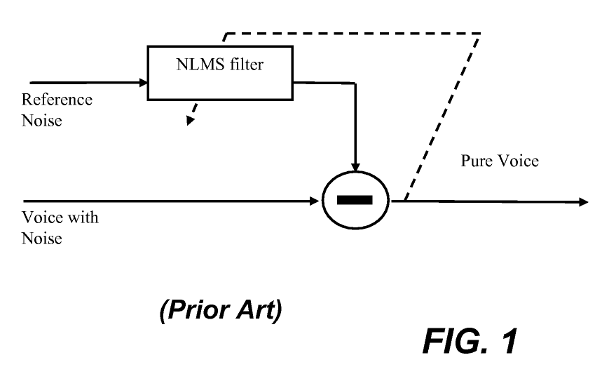

### DETAILED DESCRIPTION OF THE INVENTION

The detailed description of the present invention is presented largely in terms of procedures, steps, logic blocks, processing, or other symbolic representations that directly or indirectly resemble the operations of devices or systems contemplated in the present invention. These descriptions and representations are typically used by those skilled in the art to most effectively convey the substance of their work to others skilled in the art.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments mutually exclusive of other embodiments. Further, the order of blocks in process flowcharts or diagrams or the use of sequence numbers representing one or more embodiments of the invention do not inherently indicate any particular order nor imply any limitations in the invention.

FD-NLMS is one example of NLMS algorithm in a Frequency-Domain, whose main purpose is to reduce calculation in the NLMS adaptive filter by utilizing the frequency-domain multiplication to substitute the time-domain convolution. The detailed theory about the FD-NLMS may be found in Adaptive Filter Theory, 4^{th} Edition, by Simon Haykin

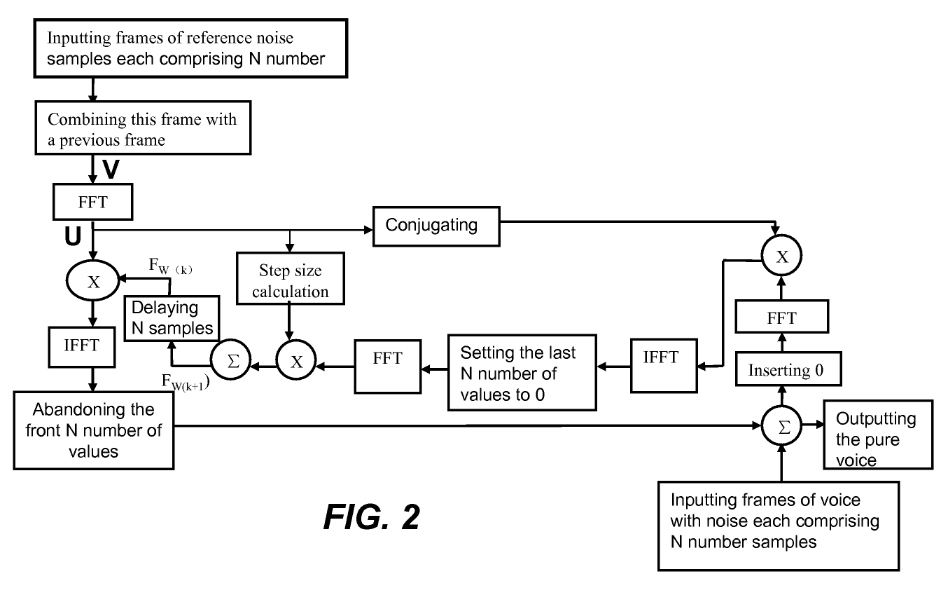

As an implementation, FIG. 2 is a flowchart of eliminating noise via an NLMS adaptive filter. The flowchart shows the following operations:

1) Inputting frames of reference noise samples, each frame has N samples; combining a current frame of reference noise samples v[0], . . . , v[N−1] with a previous frame of reference noise samples v′[0], . . . , v′[N−1] into a vector V with a size of 2N, V={v′[0], . . . , v′[N−1], v[0], . . . , v[N−1]};

2) Calculating FFT of the vector V to produce a vector U, wherein U=FFT(V)={u[0], u[1], . . . u[2N−1]} the FFT refers to Fast Fourier Transform with the size of U being 2N;

3) Multiplying the vector U by a vector Fw which is obtained from the operations 7); calculating IFFT of the product to produce a vector Y′ with the size of 2N, wherein Y′ is a real number vector (the imaginary part is 0); abandoning the front N values of Y′ to produce a vector Y, wherein Y={y[0], . . . , y[N−1]}, and its size is N;

4) Inputting frames of voice with noise simples S=s(0)K s[N−1] each frame has N samples; subtracting the vector Y from the vector S to obtain the pure voice E, wherein E={e[0], . . . , e[N−1]}={s[0]−y[0], . . . , s[N−1]−y[N−1]}.

5) Inserting N zero(0) values in the front of the vector E to form a vector E′, E′={0, . . . , 0, e[0], . . . , e[N−1]}; calculating FFT of the E′ to get a vector F, where F=FFT(E′);

6) Conjugating the U to produce a vector U^{H}, U^{H}=Ū; multiplying the vector U^{H} by the vector F to produce a vector G′; Calculating IFFT of the G′ to get a vector H, H=IFFT(G′), wherein the vector H is a real number vector with size of 2N (the imaginary part is 0); Setting the last N number of values of the vector H to 0, and then calculating FFT to the vector H to get a vector G, wherein the vector G is a plural vector with size of 2N;

7) Refreshing the vector F_{W} according to F_{W}=F_{W}+μG; wherein μ is a constant, or μ==c/Eₙ, c is a constant, Eₙ is an estimated value of the present energy of signal, F_{W} represents coefficients of the adaptive filter in FFT domain and the F_{W} calculated at this time may be regarded as the value at next time. The estimated method will not be described detailed hereafter, the detail of which may be referenced in Adaptive Filter Theory, 4th Edith, issued to Simon Haykin.

8) returning to the operation 1) until the input voice is finished.

In the method above, when calculating the pure voice in time domain, it uses the following equation:

$$e [ n ] = s [ n ] - {\sum }_{i = 0}^{N - 1} u [ n - i ] w [ i ] \qquad ( 1 )$$

wherein, s[n] stands for the value of voice with noise at the n time point; u[n] stands for the value of reference noise at the n time point; w[i] stands for coefficient of the adaptive filter; N represents the order number of the adaptive filter;

$${\sum }_{i = 0}^{N - 1} u ( n - i ) w ( i )$$

represents the estimated noise value mixed in the voice s[n] at the n time point. It can be observed that the noise may be completely eliminated from s[n] if the u[n] as the reference noise has no any voice component. However, if the reference noise u[n] contains a strong voice component, the estimated noise value

$${\sum }_{i = 0}^{N - 1} u ( n - i ) w ( i )$$

may contain a portion or all of a voice in the current frame or previous frame. As a result, the pure voice e(n) after the adaptive filter may be weakened or even introduced with echo.

An adaptive filter technique is introduced. Depending on actual implementation, the adaptive filter technique may be implemented in software or hardware. According to one embodiment of the present invention, an adaptive filter in accordance with the present invention may be advantageously used in mobile communication fields. For example, a mobile phone is provided with a pair of microphones A, B. The microphone A is positioned away from a voice source and near to a noise source (e.g., a speaker). The microphone B is positioned away from the noise source and near to the voice source. In this condition, a double track voice signal could be recorded. However, the microphones A and B simultaneously produce signals with noise due to the small size of the mobile phone and the existence of the noise source, which adversely affects the tone of the double track voice signal. It should be noted that the two adjacent voice source and noise source are taken as examples. In reality, various voice sources and noise sources coexist, thus the influence of the noise sources on the voice signals could be more serious. With one embodiment of the present invention, the influence of the noise sources on the voice signals may be minimized.

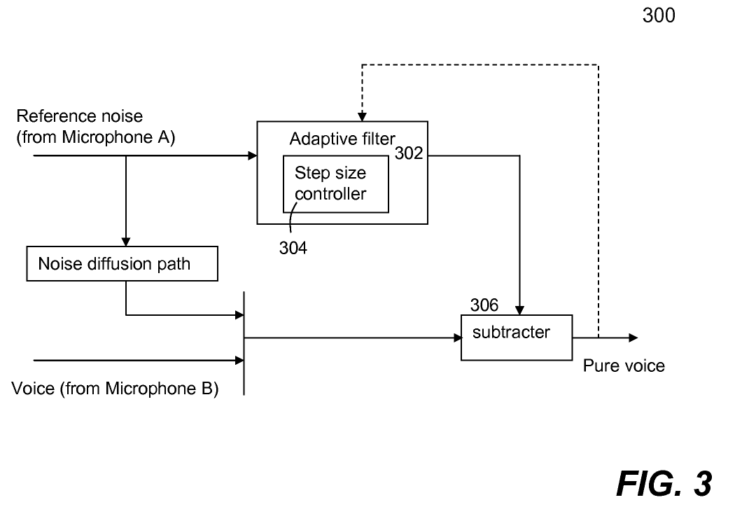

In order to minimize or eliminate the noise mixed in the voice signals, and improve the quality of the output voice, an adaptive filter method and system according to one embodiment of the present invention may be applied. It is assumed that the signal recorded by the microphone A is regarded as a voice signal with noise, and the signal recorded by the microphone B is regarded as a reference noise. FIG. 3 shows a block diagram of an adaptive filter system 300 according to one embodiment of the present invention. The adaptive filter system 300 comprises an adaptive filter 302 having a step size controller 304 and a subtracter 306. It is assumed that the order number of the adaptive filter is N, an i^{th} order of an adaptive filter coefficient is expressed as w(i).

In operation, the adaptive filter system 300 performs the following operations:

gathering frames of the voice signal s(n) mixed with noise and frames of the reference noise u(n);

inputting a current frame of the reference noise signal u(n) to the adaptive filter 302;

estimating a noise value according to the adaptive filter coefficient w(i) and the current frame of the reference noise signal u(n);

subtracting the estimated noise from the current frame of the voice signal s(n) mixed with noise to get one frame of pure voice signal e(n) by the subtracter 306;

providing the current frame of pure voice signal e(n) to the adaptive filter 302;

calculating an adaptive step size μ and estimating a voice probability contained in the current frame of reference noise, wherein μ is a constant, or μ=c/Eₙ, c is constant, Eₙ is an estimated energy of the current frame of the reference noise signal;

adjusting the adaptive step size μ according to the voice probability contained in the current frame of reference noise. Specifically, the higher the probability is, the smaller the adaptive step size μ becomes;

refreshing the adaptive filter coefficient according to the current frame of the pure voice signal, the current frame of reference noise signal, the adjusted adaptive step size μ̅; and

finally, returning to the operation of filtering a next frame of voice mixed with noise according to the refreshed adaptive filter coefficient until the voice signal mixed with noise is completely processed. Each frame of a signal comprises N number of signal simples.

In one embodiment, the refreshing operation of the adaptive filter coefficient may be performed according to the following equations:

W[k+1]=W[k]+ μ̅Σᵢ₌₀^{L-1}U(kL+i)e(kL+i);

U(kL+i)={u(kL+i),u(kL+i−1) . . . u(kL+i−N+1)};

wherein W[k+1] represents the adaptive filter coefficient at k+1 frames; L stands for refreshing the adaptive filter coefficient after L reference noise samples, L may be an integer (e.g., a multiple of N); u(kL+i) stands for the reference noise value at kL+i time point; and e[kL+i] stands for the pure voice value at kL+i time point.

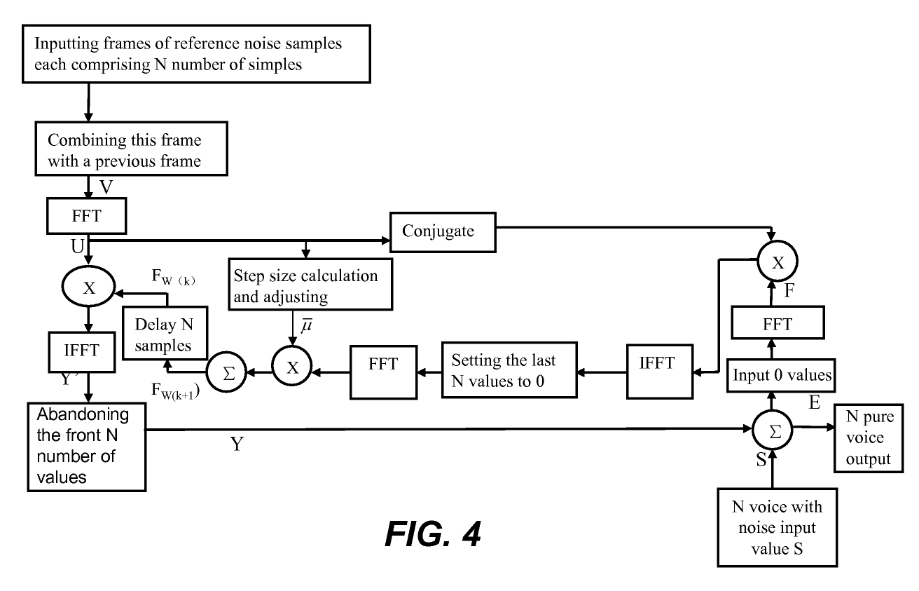

The adaptive filter operation as described may be applied into the NLMS adaptive filter algorithm. FIG. 4 shows a flowchart or process of the adaptive filter system in FD-NLMS according to one embodiment of the present invention. The adaptive filter operation according to one embodiment of the present invention includes following operations:

1) Inputting frames of reference noise samples, each frame has N samples; combining a current frame of reference noise samples v[0], . . . , v[N−1] with a previous frame of reference noise samples v′[0], . . . , v′[N−1] into a vector V with size of 2N, V={v′[0], . . . , v′[N−1], v[0], . . . v[N−1]};

2) Calculating FFT to the vector V to produce a vector U, wherein U=FFT(V)={u[0], u[1], . . . u[2N−1]}, the FFT refers to Fast Fourier Transform, the size of U is 2N;

3) Multiplying the vector U by a vector Fw which is obtained from an operation referenced as 8); calculating IFFT of the product to produce a vector Y′ with the size of 2N, wherein Y′ is a real number vector (the imaginary part is 0); abandoning the front N number of values of Y′ to produce a vector Y, wherein Y={y[0], . . . , y[N−1]}, and its size is N;

4) Inputting frames of voice with noise simples S=s(0)K s[N−1] each frame has N samples; subtracting the vector Y from the vector S to obtain the pure voice E, wherein E={e[0], . . . , e[N−1]}={s[0]−y[0], . . . , s[N−1]−y[N−1]}.

5) Inserting N number of 0 values in the front of the vector E to form a vector E′, E′={0, . . . , 0, e[0], . . . , e[N−1]}; calculating FFT to the E′ to get a vector F, F=FFT(E′);

6) Conjugating the U to produce a vector U^{H}, U^{H}=Ū; multiplying the vector U^{H} by the vector F to produce a vector G′; Calculating IFFT to the G′ to get a vector H, H=IFFT(G′), wherein the vector H is a real number vector with size of 2N (the imaginary part is 0); Setting the last N number of values of the vector H to 0, and then calculating FFT to the vector H to get a vector G, wherein the vector G is a plural vector with size of 2N;

7) calculating an adaptive step size μ and estimating a voice probability contained in the current frame of reference noise according to the vector U in the operation 2), wherein μ is a constant, or μ=c/Eₙ, c is constant, Eₙ is estimated energy of current frame of reference noise signal; adjusting the adaptive step size μ according to the voice probability contained in the current frame of reference noise, specifically, the higher the probability is, the smaller the adaptive step size μ becomes;

8) Refreshing the vector Fw according to F_{W}=F_{W}+ μ̅G; wherein F_{W} represents coefficients of the adaptive filter in FFT domain and the F_{W} calculated at this time may be regarded as the F_{W} at next time.

9) Returning to the operation 1) until the input voice is finished.

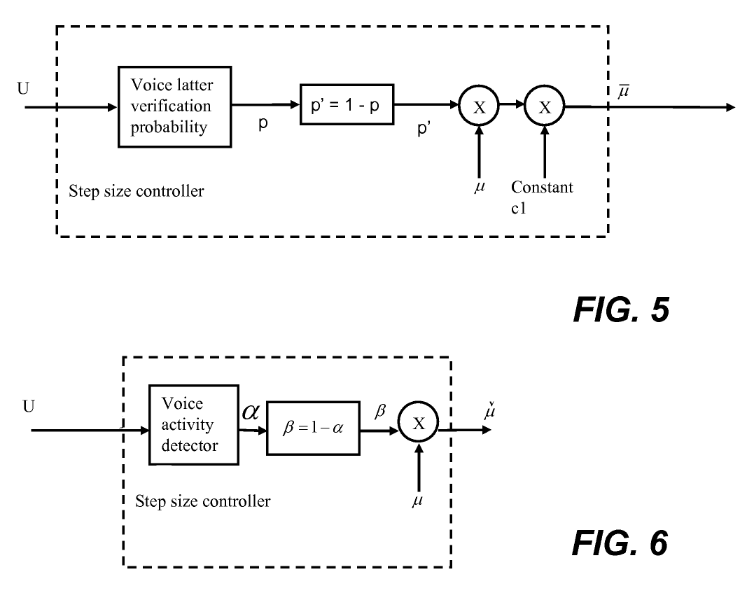

According to one embodiment, the calculating operation of the adaptive step size μ, the estimating operation of the voice probability and the adjusting operation of the adaptive step size μ are performed in a step size controller. FIG. 5 shows one example of the step size controller according to one embodiment of the present invention. As shown in FIG. 5, the voice probability p is obtained according to the vector U by a voice latter (posterior) verification probability estimation, the detail of which may be referred to in a literature named “Speech enhancement for non-stationary noise environments”, Israel Cohen, Baruch Berdugo, Signal Processing (2001), Elsevier, which is hereby incorporated by reference. Then, the adjusted adaptive step size ū can be obtained according to ū=u·C₁(1−p), wherein C₁ represents a constant.

FIG. 6 shows another example of the step size controller according to one embodiment of the present invention. As shown in FIG. 6, the voice probability α is obtained according to the vector U by a voice activity detector. Then, the adjusted adaptive step size ū can be obtained according to ū=u·(1−α).

The present invention has been described in sufficient details with a certain degree of particularity. It is understood to those skilled in the art that the present disclosure of embodiments has been made by way of examples only and that numerous changes in the arrangement and combination of parts may be resorted without departing from the spirit and scope of the invention as claimed. Accordingly, the scope of the present invention is defined by the appended claims rather than the foregoing description of embodiments.

We claim:

1. A method for an adaptive filter, the method comprising:
gathering frames of a voice signal mixed with noise and frames of a reference noise signal;
estimating a noise value according to a current coefficient of the adaptive filter and a current frame of a reference noise signal;
subtracting the estimated noise value from a current frame of the voice signal mixed with noise to produce a frame of a pure voice signal;
calculating an adaptive step size and estimating a voice probability contained in the current frame of the reference noise;
adjusting the adaptive step size according to the voice probability;
refreshing the adaptive filter coefficient according to the current frame of pure voice signal, the current frame of reference noise signal, and the adjusted adaptive step size.

2. The method as claimed in claim 1, wherein said adjusting the adaptive step size is performed according to an equation expressed as:
ū=u·(1−α)
wherein α represents the voice probability, and ū represents the adjusted adaptive step size.

3. The method as claimed in claim 1, wherein said adjusting the adaptive step size is performed according to an equation expressed as:
ū=u·C₁(1−p),
wherein C₁ is a constant, p represents the voice probability, and ū represents the adjusted adaptive step size.

4. The method as claimed in claim 1, wherein said estimating a voice probability contained in the current frame of the reference noise is performed by a voice activity detector.

5. The method as claimed in claim 1, wherein said estimating a voice probability contained in the current frame of the reference noise is performed by a voice latter (posterior) verification probability estimation.

6. The method as claimed in claim 1, wherein said refreshing the adaptive filter coefficient is performed according to an equation expressed as follows:
F_{W}=F_{W}+ μ̅G
wherein F_{W} represents the adaptive filter coefficient, ū represents the adjusted adaptive step size, and G represents the current frame of pure voice signal and the current frame of reference noise signal.

7. The method as claimed in claim 1, wherein the method is applied in mobile communication equipments.

8. The method as claimed in claim 1, wherein the voice signal mixed with noise is obtained from a first microphone, and the reference noise signal is obtained from a second microphone, both of the first and second microphones are located adjacently on a mobile phone.

9. An adaptive filter comprising:
an adaptive filter for receiving frames of a reference noise signal, estimating a noise value according to a current coefficient of the adaptive filter and a current frame of the reference noise signal;
a subtracter for receiving frames of a voice signal mixed with noise and subtracting the estimated noise value from a current frame of the voice signal mixed with noise to produce one frame of pure voice signal;
an step size controller for calculating an adaptive step size, estimating a voice probability contained in the current frame of reference noise, and adjusting the adaptive step size according to a voice probability, wherein the adaptive filter refreshes the adaptive filter coefficient according to the current frame of the pure voice signal, the current frame of reference noise signal and the adjusted adaptive step size.

10. The adaptive filter as claimed in claim 9, wherein the adaptive step size in the step size controller is performed according to an equation expressed as:
ū=u·(1−α)
wherein α represents the voice probability, and ū represents the adjusted adaptive step size.

11. The adaptive filter as claimed in claim 9, wherein the adaptive step size in the step size controller is performed according to an equation expressed as:
ū=u·C₁(1−p),
wherein C₁ is a constant, p represents the voice probability, and ū represents the adjusted adaptive step size.

12. The adaptive filter as claimed in claim 9, wherein the step size controller comprises a voice activity detector for estimating the voice probability.
